(19) United States
(12) Patent Application Publication
(10) Pub. No.: US 2004/0220198 A1
(43) Pub. Date: Nov. 4, 2004

(54) Methods for treating malaria by modulation of G protein function

(76) Inventors: Kasturi Haldar, Chicago, IL (US); Travis Harrison, Chicago, IL (US); Jon Lomasney, Oak Park, IL (US); Heidi Hamm, Nashville, TN (US)

Correspondence Address: KLAUBER & JACKSON, 411 HACKENSACK AVENUE, HACKENSACK, NJ, 07601

(21) Appl. No.: 10/740,368

(22) Filed: Dec. 18, 2003

Related U.S. Application Data
(60) Provisional application No. 60/434,915, filed on Dec. 20, 2002.

(51) Int. Cl. A61K 31/522; A61K 31/137

(52) U.S. Cl. 514/263.31; 514/651

(57) ABSTRACT

The present invention is directed to methods and pharmaceutical compositions for treating a mammal suffering from malaria or the sequelae of malarial infection, or for preventing a malarial infection, or for ameliorating the symptoms associated with a malarial infection using a therapeutically effective amount of an agent that down regulates G protein mediated functions. Also contemplated are methods for screening for novel compounds that down regulate G protein receptors, and the use of these compounds for treating mammals having malaria.

### CROSS-REFERENCE TO RELATED APPLICATIONS

The present application is a non-provisional application claiming the priority of provisional application Serial No. 60/434,915, filed Dec. 20, 2002, the disclosure of which is incorporated by reference herein in its entirety. Applicants claim the benefits of this application under 35 U.S.C. §119 (e).

### GOVERNMENT RIGHTS CLAUSE

The research leading to the present invention was supported by Grant No. AI39071. Accordingly, the Government has certain rights in the invention.

### FIELD OF THE INVENTION

The present invention is directed to methods and pharmaceutical compositions for treating a mammal suffering from malaria by administering a therapeutically effective amount of a compound that down regulates G protein mediated functions in host cells susceptible to malarial infection.

### BACKGROUND OF THE INVENTION

Malaria is a disease that continues to have an impact in much of the developing world. This disease, which afflicts 200-300 million people, results in considerable morbidity (eg. fever and chills, malaise, anorexia, kidney disease and brain disease) and kills over one million children each year. The intracellular protozoa, Plasmodium falciparum, is the most virulent of human malarias and accounts for greater than 95% of malarial deaths. High levels of parasites in the bloodstream, seen especially in the P. falciparum infection, causes serious complications including severe hemolytic anemia, renal failure, and coma. Thus, diagnosis and early treatment of P. falciparum is crucial.

An important contributor to the increase in incidence of malaria over the past 30 years has been the development of resistance of the malarial parasite to existing drugs. For example, chloroquine resistance is widespread, partial resistance to quinine is seen in many areas, and resistance to the combination of pyrimethamine and sulfadoxine has been reported (Eyles et al. 1963, Am. J. Trop. Med. Hyg. 12, 840-835; WHO Tech. Report Series No. 711, 1984; Boudreau et al. 1982 Lancet II, 1335; Noreen et al. 1991, Lancet 337, 1140-1143; Hurwitz et al. 1981, Lancet I, 1068-1070; Timmermanns et al 1982, Lancet I 11181). Although quinine resistance is also emerging along the Thailand-Myanmar border, still the quinine and tetracycline combinations remain over 80% effective in practice (Vanijanonta et al. 1992, Lancet 339,369). Mefloquine is a new anti-malarial that may be effective against chloroquine-resistant P. falciparum. However, Mefloquine is associated with some undesirable side effects. In particular, mefloquine has been reported to produce neuropsychiatric side-effects in adults who developed tonic clonic fits. Besides psychosis, delusions and hallucinations, anxiety sleep disturbances were also reported after treatment with mefloquine (Panisko D M and Keystone J S, Drugs 1990, 39, 160-169). Since treatment failure may occur with any drug regimen, the course of the parasitemia must be followed closely. The non-falciparum parasites are usually treated with chloroquine or amodiaquine, followed by treatment with primaquine if infection is caused by P. vivax or P. ovale. Halofantrine is more effective; however, rather high doses of the drug are now required to control resistant P. falciparum (Brasseur et al. 1993. Lancet 341, 901-2), doses reportedly that could lead to increased risk of cardiotoxicity of this new antimalarial, including sinus bradycardia, sinus arrhythmia, tall peak T. waves, QT interval prolongation, or ectopic beats (Karbwang et al. 1993, Lancet, 342, 501; Wildling et al. 1993. Lancet, 342, 55; Kremsner el al. Am. J. Trop. Med. Hyg. 50, 790-795). These findings, unfortunately, have imposed great limitations on the antimalarial potential of this drug. Several reports have recently appeared which document emergence of chloroquine resistance by P. vivax (Schwartz et al. 1991. New England J. Med. 324; Schuurkamp et al. 1992. Trans. R. Soc. Trop. Med. Hyg. 86, 121-2; Murphy el al., 1993. Lancet, 341, 96-100; Garg et al. 1995. Trans. R. Soc. Trop. Med. Hyg. 89, 656-7; Marlar-Than et al. 1995. Trans. R. Soc. Trop. Med. Hyg. 89, 307-8; Baird et al., 1996. Trans. R. Soc. Trop. Med. Hyg. 90, 409-411, Baird et al., 1997, Am. J. Trop. Med. Hyg. 56, 627-631. The World Health Organization (1984) had accorded high priority to the development of fast acting artemisinin derivatives as blood schizontocides for the emergency treatment of cerebral malaria as well as for the control of multiple drug resistant cases of Plasmodium falciparum.

The asexual blood stages of infection are responsible for all of the symptoms and pathologies associated with the disease. Blocking these stages is expected to be important for controlling acute infection as well as disease pathologies. Thus, development of new drugs that are efficacious against blood stage infection is critical to disease control. The asexual blood stage parasite infects the mature red cell. Mature red cells are unusual host cells in that they are terminally differentiated, devoid of all intracellular organelles, incapable of de novo protein or lipid synthesis and lack endocytic machinery (Chasis et al., 1989). P. falciparum infects the red blood cell and develops enclosed within a parasitophorous vacuolar membrane (PVM) inside the cell. Furthermore, the intravacuolar parasite alters antigenic and transport properties of the red cell (Deitsch and Wellems, 1996).

The tubovesicular membrane network (TVN) that emerges from the PVM and extends to the periphery of the red cell (Elford and Ferguson, 1993; Elmendorf and Haldar, 1994; Haldar, 1998)) provides the major pathway to deliver both host proteins and extracellular nutrients to the plasmodial vacuole (Akompong et al., 1999a; Lauer et al., 1997; Lauer et al., 2000). The significance of parasite-induced modifications of the red cell lies in mechanisms of vacuolar transport that are thought to be critical for malarial survival in blood.

It is well established that G proteins are coupled to receptors and mediate a number of signaling events in a wide variety of cells. However, G protein function in mature red blood cells is poorly studied. The red cell is enucleated, has no intracellular structures, and is incapable of de novo protein and lipid biosynthesis. Thus, the requirement for G protein function in this cell remains unclear. Since little has been done with respect to the identification of new therapeutic agents for treatment of malaria, and due to the emergence of resistance of the parasite to existing therapies, it is to the potential role of G protein mediated function on infection of red blood cells by the malarial parasite and the identification of a potential new target for development of anti-malarial therapies that the present invention is directed.

### SUMMARY OF THE INVENTION

In its broadest aspect, the invention relates to the identification of agents that down-regulate G protein mediated signaling in red blood cells susceptible to infection by malaria, while at the same time exhibiting a deleterious effect on infection of the host cell by the parasite responsible for the malarial infection, Plasmodium falciparum. It is a further object of the invention to demonstrate that interfering with Gs-G-protein coupled receptor interactions blocks malarial infection. None of the presently available antimalarial drugs are targeted against G proteins. Thus, it is a yet further object of the invention to provide evidence that inhibition of G protein function provides a new approach to treat parasites resistant to existing drugs. The potential use of drugs that down regulate G protein mediated function for treatment of malaria was not realized until the time of the present invention.

Accordingly, a first aspect of the invention provides for a method of treating a mammal suffering from malaria or the sequelae of malarial infection, comprising administering a therapeutically effective amount of a compound that down-regulates G protein signaling, in cells susceptible to infection by malaria.

In a preferred embodiment, the method for treating comprises administration of a compound that is an antagonist of a G protein receptor. In another preferred embodiment, the receptor is a β-adrenergic receptor or an adenosine receptor. In yet another preferred embodiment, the antagonists may be selected from the group consisting of 8-p-sulfophenyltheophylline (8-SPT), Acebutolol, Atenolol, Betxolol, Bisoprolol, Esmolol, Metoprolol, Carteolol, Nadolol, Penbutolol, Pindolol, Propranolol, Sotalol, Timolol, Carvedilol, Labetalol, Alprenolol, and ICI 118,551.

A second aspect of the invention provides for a method for preventing a malarial infection in a mammal comprising administering a therapeutically effective amount of a compound that down-regulates G protein signaling in cells susceptible to infection by malaria.

In a preferred embodiment, the method for preventing a malarial infection in a mammal comprises administration of a compound that is an antagonist of a G protein receptor. In another preferred embodiment, the receptor is a β-adrenergic receptor or an adenosine receptor. In yet another preferred embodiment, the antagonists may be selected from the group consisting of 8-p-sulfophenyltheophylline (8-SPT), Acebutolol, Atenolol, Betxolol, Bisoprolol, Esmolol, Metoprolol, Carteolol, Nadolol, Penbutolol, Pindolol, Propranolol, Sotalol, Timolol, Carvedilol, Labetalol, Alprenolol, and ICI 118,551.

A third aspect of the invention provides for a method for ameliorating the symptoms or the anemia or the sequelae associated with a malarial infection in a mammal comprising administering a therapeutically effective amount of a compound that down-regulates G protein signaling in cells susceptible to infection by malaria.

In a preferred embodiment, the method for ameliorating the symptoms associated with a malarial infection in a mammal comprises administration of a compound that is an antagonist of a G protein receptor. In another preferred embodiment, the receptor is a β-adrenergic receptor or an adenosine receptor. In yet another preferred embodiment, the antagonists may be selected from the group consisting of 8-p-sulfophenyltheophylline (8-SPT), Acebutolol, Atenolol, Betxolol, Bisoprolol, Esmolol, Metoprolol, Carteolol, Nadolol, Penbutolol, Pindolol, Propranolol, Sotalol, Timolol, Carvedilol, Labetalol, Alprenolol, and ICI 118,551.

A fourth aspect of the invention provides for methods of treating a mammal suffering from malaria or the sequelae of malarial infection, methods for preventing a malarial infection, or methods of ameliorating the symptoms associated with a malarial infection in a mammal comprising administration of a combination of compounds that down-regulate G protein signaling in cells susceptible to infection by malaria. In a preferred embodiment, the methods comprise administration of at least one compound that is an antagonist of a G protein receptor with a second compound that is also an antagonist of a G protein receptor. In another preferred embodiment, these antagonists may be selected from the group consisting of 8-p-sulfophenyltheophylline (8-SPT), Acebutolol, Atenolol, Betxolol, Bisoprolol, Esmolol, Metoprolol, Carteolol, Nadolol, Penbutolol, Pindolol, Propranolol, Sotalol, Timolol, Carvedilol, Labetalol, Alprenolol, and ICI 118,551. In a yet further preferred embodiment, the methods may employ administration of one or more compounds that downregulate G protein signaling, such as the antagonists of a G protein receptor described above, in conjunction with one or more anti-malarial compounds such as those commonly utilized to treat malarial infections. These anti-malarial compounds may be selected from the group consisting of chloroquine, quinine, mefloquine, amodiaquine, primaquine, pyrimethamine, sulfadoxine, sulfadiazine, trimethoprim, pentavalent antimony, pentamidine, amphotericin B, rifampin, metronidazole, ketoconazole, benznidazole and nifurtimox.

A fifth aspect of the invention provides for pharmaceutical compositions comprising a therapeutically effective amount of at least one compound that down regulates G protein signaling in cells susceptible to infection by malaria and a pharmaceutically acceptable carrier. Such pharmaceutical compositions can be targeted to parasite infected red blood cells. As is well known in the art, parasite-encoded adhesion molecules are inserted into the erythrocyte membrane. These molecules are encoded by malaria genes called variable adhesion proteins, or VAR. In one embodiment, antibodies against these molecules can be utilized to target therapies as described herein to the red blood cells. Anrews et al. 2003 Mol. Microbiol. 49:655-69; Giha et. al., 2000, Immunol. Lett. 71: 117-26. In a preferred embodiment, the composition is administered orally, in the form of a tablet or capsule. In a yet further preferred embodiment, the composition is administered in the form of a sustained release formulation.

A sixth aspect of the invention provides for a method of screening for compounds that act as antagonists to the Gs coupled receptor on red blood cells that are susceptible to malarial infection. Accordingly, it is yet a further object of the invention to screen for compounds useful for treating or preventing a malarial infection in a mammal or for ameliorating the symptoms associated with malarial infection in a mammal comprising contacting a preparation containing the cells bearing these receptors with a test compound or a control compound and determining the ability of the test compound to modulate (e.g., stimulate or inhibit) the expression or activity of the receptor. Such methods are well known in the art. For example, the methods disclosed in the following are well known techniques to screen for compounds that act as antagonists to Gs coupled receptors: Barak L S, Ferguson S S, Zhang J, Caron M G. (1997) J Biol Chem. October 31; 272(44):27497-500, or disclosed in Barak L S, Zhang J, Ferguson S S, Laporte S A, Caron M G. (1999) ;302: 153-71. Furthermore, techniques as disclosed in Yves Durocher, 1 Sylvie Perret, Eric Thibaudeau, Marie-Helene Gaumond, Amine Kamen, Rino Stocco, and Mark Abramovitz. (2000) Analytical Biochemistry 284, 316-326 also provide well known methods for the screening described herein.

The compounds identified as inhibitors/antagonists of G protein signaling can then be used to treat mammals suffering from malaria or the sequelae of malarial infection. These compounds can also be used to prevent malarial infection in a mammal comprising administering a therapeutically effective amount of a compound that down regulates G protein signaling. Such compounds can also be used to ameliorate symptoms associated with a malarial infection in a mammal. The novel agents identified by the methods described herein may be used alone in the treatment of malaria, or they may be used as adjunct therapy with other agents to treat malaria, or they may be used to prevent malaria or ameliorate the symptoms of malaria, alone or in conjunction with other anti-malarial agents. The instant invention also provides for pharmaceutical compositions comprising the novel compounds identified by the screening methods described herein.

Other advantages of the present invention will become apparent from the ensuing detailed description taken in conjunction with the following illustrative drawings.

### BRIEF DESCRIPTION OF THE DRAWINGS

FIG. 1. Effects of preincubating (for 4 h) red cells or schizonts with Gs peptide on malarial infection. Uninfected red cells or schizonts were resuspended at 2×10⁸ cells/ml in RPMI1640 and incubated with 500 μM Gs peptide or mock treated for 4 h at 37° C. The cells were subsequently recovered by centrifugation and washing in peptide-free medium. Washed cells were then subjected to an overnight infection assay (using 4% schizonts to infect the culture) in the absence of peptides.

FIG. 2. Effects of FITC-Gs peptide and its FITC-Gs-scrambled peptide on erythrocytic infection. Assays containing 500 μM of indicated peptides, their FITC-derived counterparts or solvent alone were added to a standard infection assay (see Table 1).

FIG. 3. Cells taken from a standard infection assay incubated with 500 μM FITC-Gs peptide (A) or FITC-Gs scrambled peptide (B) were fixed in formaldehyde, and without permeabilization subjected to an indirect immunofluorescence assay with anti-MSP1 antibody (red, to detect extracellular parasites) and stained with DAPI (blue, to visualize nuclei). Samples were imaged by digitized fluorescence microscopy (FITC signal is green).

FIG. 4. Model of peptide translocation into infected cells. A schematic drawing of Gαs inhibition of ring formation. On the basis of data in FIG. 3, the applicants propose that the peptide is taken in with the parasite and is translocated across the nascent or newly formed vauole. Presence of the FITC-Gαs peptide prevented intracellular ring formation, whereas FITC-Gαscr allows intracellular ring formation.

FIG. 5. Effects of β-adrenergic receptor agonist (isoproterenol) or antagonist (propranolol) on erythrocyte infection by P. falciparum. Cultures containing 2×10⁸ red cells and 2.5×10⁶ synchronized segmenters were incubated in 1 ml of 10% medium (10% human serum with RPMI) with the indicated agonist, antagonist, agonist+antagonist, or mock treated (solvent alone: 1 μl) for 6 h. Virtually no schizonts remained at this time and the cultures contained new intracellular rings as detected by Giemsa staining. The lower panel indicates percent change compared to mock treated cultures.

FIG. 6. Effects of Gs peptide on isoproterenol-stimulated infection of erythrocytes by P. falciparum. Cultures treated with isoproterenol (as described in FIG. 5) for 6 h in the presence of 200 μM Gs peptide or Gs scrambled peptide and scored for new ring formation (see FIG. 5). Lower panel indicates percent change compared to mock treated cultures.

FIG. 7. Effects of adenosine receptor agonist (NECA) and antagonist (8-SPT) on infection of erythrocytes by P. falciparum. Cultures were set up as described in FIG. 5, indicated concentrations of NECA, 8-SPT, both or solvent alone were added and infection was scored after 6 h as described.

FIG. 8. Stimulatory effect of adenosine receptor agonist adenosine, on plasmodial infection and its inhibition by Gs peptide. Cultures were set up as described in FIG. 5 incubated with the indicated levels of adenosine in the presence of (200 μM) Gs peptide, Gs scrambled peptide or no peptide. New ring infection was scored after 6 h as described (FIG. 5).

FIG. 9. Effects of (β-adrenergic and adenosine receptor) agonists and antagonists singly and in combination on in vitro erythrocytic infection by P. falciparum. In vitro infection assays were done with synchronized cultures of P. falciparum (strain 3D7) using standard culture conditions (K. Haldar, M. A. J. Ferguson, G. A. M. Cross, J Biol Chem 260, 4969 (1985)). A starting parasitemia of 2.5% schizonts (44-48 h in development) in 20 μl of red blood cells with 1 ml of 10% human serum in RPMI1640 was used. The cultures were incubated for 4-6 hours in the presence of Gαs protein receptor agonist or antagonist, or vehicle control. All detectable schizonts ruptured, and new ring stage infection was scored by Giemsa staining of thin blood smears. In all experiments, control cultures achieved ring parasitemias of 8-11%, and standard error was 10%.

FIG. 10. In vitro effect of various β-adrenergic antagonists on inhibition of P. falciparum infection in red blood cells treated with isoproterenol. In vitro infection assays were done with synchronized cultures of P. falciparum (strain 3D7) using standard culture conditions (K. Haldar, M. A. J. Ferguson, G. A. M. Cross, J Biol Chem 260, 4969 (1985)). A starting parasitemia of 2.5% schizonts (44-48 h in development) in 20 μl of red blood cells with 1 ml of 10% human serum in RPMI1640 was used. The cultures were incubated for 4-6 hours in the presence of 10 μM beta-adrenergic receptor agonists or antagonists, or vehicle control. All detectable schizonts ruptured, and new ring stage infection was scored by Giemsa staining of thin blood smears. In all experiments, control cultures achieved ring parasitemias of 8-11%, and standard error was 10%.

FIG. 11. The effect of the racemic form of propranolol on growth of P. berghei in vivo. In vivo mouse experiments were done with P. berghei using a standard 4 day Peters assay (W. Peters, B. L. Robinson, Ann Trop Med Parasitol 78, 561 (1984)). P. berghei and the racemic form of propranolol were administered intraperitoneally; five mice were used per data point. In IC₅₀ studies, 5 data points were taken. Mice were given 5×10⁷ parasites on day 0, and indicated concentrations of drug twice a day on days 0-3. Tail bleeds were carried out on day 4 to ascertain parasitemia, after which the animals were sacrificed.

FIG. 12. The effect of ICI 118,551 on growth of P. berghei in vivo. In vivo mouse experiments were done with P. berghei using a standard 4 day Peters assay (W. Peters, B. L. Robinson, Ann Trop Med Parasitol 78, 561 (1984)). P. berghei and ICI 181,551 were administered intraperitoneally; five mice were used per data point. In IC₅₀ studies, 5 data points were taken. Mice were given 5×10⁷ parasites on day 0, and indicated concentrations of drug twice a day on days 0-3. Tail bleeds were carried out on day 4 to ascertain parasitemia, after which the animals were sacrificed.

FIG. 13. Comparison of the antagonists racemic propranolol, ICI, altenolol and nadolol in vivo using P. berghei.

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated herein by reference.

### Definitions

“Treatment” refers to the administration of medicine or the performance of medical procedures with respect to a patient, for either prophylaxis (prevention) or to cure the infirmity or malady in the instance where the patient is afflicted.

A “therapeutically effective amount” is an amount sufficient to prevent the disease or to decrease or ameliorate the symptoms associated with the malarial infection.

An “agent”, as used herein refers to all materials that may be used to prepare pharmaceutical and diagnostic compositions, or that may be compounds, nucleic acids, polypeptides, fragments, isoforms, variants, or other materials that may be used independently for such purposes, all in accordance with the present invention.

“Combination therapy” refers to the use of the agents of the present invention with other active agents or treatment modalities, in the manner of the present invention for the prevention or treatment of malaria or the sequelae associated with a malarial infection. These other agents or treatments may include drugs such as other antagonists of G protein signaling or they may be other standard anti-malarial drugs. The combined use of the agents of the present invention with these other therapies or treatment modalities may be concurrent, or the two treatments may be divided up such that the agent of the present invention may be given prior to or after the other therapy or treatment modality.

The term “G protein” is meant any of a family of similar heterotrimeric (ie. made up of three different subunits) proteins of the intracellular portion of the plasma membrane that bind receptor complexes and, through conformational changes and cyclic binding and hydrolysis of GTP, couple cell surface receptors to intracellular responses. The three subunits are Gα, which carries the binding site for the nucleotide, Gβ, and Gγ. In the inactive state, Gα has GDP in its binding site. When a hormone or other ligand binds to the associated G protein coupled receptor, an allosteric change takes place in the receptor. This triggers an allosteric change in Gα causing GDP to leave and be replaced by GTP. GTP activates Gα causing it to dissociate from GβGγ (which remained linked as a dimer). Activated Gα in turn activates an effector molecule, such as, for example, adenylyl cyclase. One type of Gα subunit is designated Gαₛ (for stimulatory). This type stimulates adenylyl cyclase and thus increases the level of cAMP in the cell. Others such as Gα_{q} activates phospholipase C (PLC) and generates the second messengers inositol triphosphate and diacylglycerol. Gαᵢ are subunits that inhibit adenylyl cyclase thereby lowering the level of cAMP in the cell. One example of this type of subunit is Gαₜ for transducin, the molecule responsible for generating a signal in the rods of the retina in response to light. It also triggers the breakdown of cGMP.

“G protein coupled receptors” or “GPCR” are transmembrane proteins that wind 7 times back and forth through the plasma membrane. Their ligand-binding site is exposed outside the surface of the cell. Their effector site extends into the cytosol. The ligand binds to a site or sites on the extracellular portion of the receptor. This binding activates a G protein associated with the receptor's cytoplasmic C-terminal. This initiates the production of a second messenger, the most common of which are cAMP produced by adenylyl cyclase, and inositol 1,4,5-triphosphate (IP₃). The second messenger initiates a series of intracellular events such as phosphorylation and activation of enzymes, and release of Ca²⁺ from within the cytoplasm. In the case of cAMP, in nucleated cells, these enzymatic changes activate the transcription factor CREB (cAMP Response Element Binding protein). Once bound to its response element, activated CREB turns on gene transcription. The cell therefore begins to produce the appropriate gene products in response to the signal it had received at its surface. Thus, the function of the GPCR is to interact with G-proteins to transduce signals that induce a cellular response to the environment.

“G protein function” relates to the interactions of G-proteins with G-protein coupled receptors and the ability of G-proteins to modulate second messenger activity within a cell through conformational changes and binding and hydrolysis of GTP. For example, as related to the present invention, the antagonists that demonstrate the desired activity in terms of prevention of malarial parasitic growth within the red blood cell as upon antagonist binding to G-protein coupled receptors within the red blood cell, cAMP production is decreased.

By the term “sequelae” of a malarial infection is meant the conditions following as a consequence of the malarial infection or disease. This may include fever and chills, malaise, anorexia, kidney disease and brain disease. Patients having malaria also experience asymptomatic parasitemia; acute febrile illness (with cerebral damage, anemia, respiratory distress, hypoglycemia); chronic debilitation (anemia, malnutrition, nervous system-related disorders such as cognitive impairment); and complications of pregnancy (anemia, low birth weight, increased infant mortality).

The term “downregulates” as used herein refers to a decrease in the expression and/or function of the G protein receptor and subsequent signaling events initially triggered by the binding of the ligand with the receptor. For example, a downregulated β-adrenergic receptor may be unable to induce Gαs protein signaling, or if β-adrenergic receptors are downregulated the total number of β-adrenergic receptors expressed in a cell is decreased. The down-regulated G protein receptor function may also refer to a decrease in signaling capacity following incubation of cells bearing G protein receptors with specific inhibitory or blocking agents as described herein, such that signaling function is then abrogated.

An “agonist” is an endogenous substance or a drug that can interact with a receptor and initiate a physiological or a pharmacological response characteristic of that receptor (contraction, relaxation, secretion, enzyme activation, etc.). An agonist has a positive intrinsic activity. “Intrinsic activity” is the the ability of a drug (and cell) to transduce a drug-receptor binding event into a biological response.

“β-adrenergic receptor antagonists” are a class of drugs that compete with beta-adrenergic agonists for available receptor sites; some compete for both β1- and β2-adrenergic receptors (e.g., propranolol) while others bind primarily to either β1- (e.g., metoprolol) or β2-adrenergic receptors; these compounds are used in the treatment of a variety of cardiovascular diseases where beta-adrenergic blockade is desirable. Antagonists have an intrinsic activity of zero. These agents are also called beta-adrenergic receptor blocking agents, or beta-adrenoreceptor antagonists. They are also known as beta-blockers. Examples of these agents include Acebutolol (N-[3-Acetyl-4-[2-hydroxy-3-[(1-methylethyl)amino]phenyl]butamamide), Atenolol (4-[2-Hydroxy-3-[(1-methylethyl)amino]-propoxy]benzeneacetamide) , Betaxolol (1-[4-[2-(cyclopropylmethoxy)ethyl]-phenoxy]-3-[(1-methylethyl)amino]-2-propanolol), Bisoprolol (1-[4-[(2-(1-methylethoxy)ethoxy)methyl)phenoxy]-3-[(1-methylethyl)amino]-2-propanolol), Esmolol (Methyl-4-[2-hydroxy-3-[1-methylethyl)amino]-propoxy]benzenepropanoate), Metoprolol (1-[4-(2-Methoxyethyl)phenoxy]-3-[1-methylethyl)amino]-2-propanol, Carteolol (5-[3-[(1,1-Dimethylethyl)amino]-2-hydroxypropoxy]-3,4-dihydro-2(1H)-quinolinone), Nadolol (5-[3-[(1,1-Dimethylethyl)amino]-2-hydroxypropoxy]-1,2,3,4-tetrahydro-2,3-naphthalenediol, Penbutolol (1-(2-Cyclopentylphenoxy)-3-[1,1-dimethylethyl)amino]-2-propanol), Pindolol (1-(1H-Indol-4-yloxy)-3-[1-methylethyl)amino]-2-propanol), Propranolol (1-[(1-Methyleth -3-(1-naphthalenyloxy)-2-propanol), Sotalol (N-[4-[1-Hydroxy-2-[(1-methylethyl)amino]ethyl]phenyl]methanesulfonamide), Timolol (1-[(1,1-Dimethylethyl)amino]-3-[[4-morpholinyl-1,2,5-thiadizaol-3-yl]oxy]-2-propanol), Carvedilol (1-(Carbazol-4-yloxy)-3-[[2-(O-methoxyphenoxy)ethyl]amino]2-propanol), Labetalol (2-Hydroxy-5-[1-hydroxy-2-{(1-methyl-3-phenylpropyl)amino]ethyl]benzamide), Alprenolol (1-[(Methylethyl)amino]-3-[2-(2-propenyl)phenoxy]-2-propanol, and ICI 118,551.

“Adenosine receptor antagonists” are drugs that compete with adenosine receptor agonists for available receptor sites. Antagonists have an intrinsic activity of zero. One example of such an antagonist is 8-SPT. Adenine nucleosides and nucleotides have multiple effects as extracellular mediators in every organ system and initiate or modulate cellular responses via cell surface receptors. Current evidence indicates the existence of four receptors for adenosine: A₁, A_{2A}, A_{2B}, and A₃ (Fredholm, B. B., ET AL., (1998). Adenosine receptors. In The IUPHAR Compendium of Receptor Characterization and Classification. IUPHAR Media Ltd., Cambridge. 48-57). These G protein-coupled receptors transduce activation or inhibition of adenylate cyclase and phospholipase C. Reasonably selective antagonists are available for some adenosine receptor subtypes. Additional information about the molecular characteristics of these receptors, their pharmacologic properties, and associated signaling pathways can be found in several recent compendia and reviews (Dubyak, G. R., and J. S. Fedan, editors. (1990), Biological Actions of Extracellular ATP, The New York Academy of Sciences, New York; Harden, T. K., ET AL. (1995), P₂-purinergic receptors: subtype-associated signaling responses and structure. Annu. Rev. Pharmacol. Toxicol. 35: 541-579)

The term “inverse agonist” as used herein refers to a compound that produces conformational changes in the receptor that are less favorable to activation of G-protein coupled receptors than the ground state. An inverse agonist is a compound with a negative intrinsic activity. An inverse agonist is also called a negative antagonist.

### General Description

As described herein, signaling via erythrocyte β-adrenergic receptors and heterotrimeric G protein Gαs regulates the entry of the human malaria parasite Plasmodium falciparum into the cell. In particular, it has been shown that agonists that stimulate cAMP production lead to an increase in malarial infection, which can be blocked by specific receptor antagonists.

Moreover, peptides designed to inhibit Gαs protein function reduce parasitemia in P. falciparum cultures in vitro and β-antagonists reduce parasitemia of a P. berghei infection in an in vivo mouse model. Thus, signaling via the erythrocytic β-adrenergic receptor and Gαs may regulate malarial infection across species. These data suggest that β-blockers, drugs commonly used to treat hypertension, may show promise as effective anti-malarials. They may be particularly useful in combination with existing anti-malarials directed against parasite targets. The studies provided herein have aimed to limit rapidly emerging resistance to conventional anti-parasitic drugs.

### Therapeutic Uses of the Invention

As described herein, the establishment of infection in a red cell by the human malaria parasite Plasmodium falciparum is retarded by agents that down regulate G protein mediated functions in the host cell, in particular signaling, thereby providing evidence for red cell G protein function in malarial infection.

Accordingly, one aspect of the current invention is a method for treating a mammal suffering from malaria or the sequelae of malarial infection, or preventing malarial infection in a mammal, or ameliorating the symptoms associated with a malarial infection, comprising administering a therapeutically effective amount of a compound that down-regulates G protein function in cells susceptible to infection by malaria. The therapeutic methods envisioned by the present invention include use of at least one antagonist of a G protein receptor. However, it is also believed that a combination of at least two G protein receptor antagonists may be beneficial. It is also believed that the agents of the present invention may also be combined with other standard anti-malarial therapies known to those skilled in the art.

The inventors of the instant application have taken advantage of targeting the host cell which is not as likely to become resistant to drug therapy as rapidly as the parasite target itself. If compounds against the host target act synergistically with existing anti-parasitic drugs, they may reduce required levels of existing drugs and restore the use of drugs to which there is presently resistance. In the matter of the present invention, the compounds that have been identified are β-blockers, a well established class of drugs, that are well tolerated, easily available, inexpensive and could be rapidly used to develop a new generation of anti-malarials. Neither this approach nor the compounds have previously been tested for this utility.

The inventors of the present application have investigated the presence and contents of detergent resistant membrane (DRM) lipid rafts in mature human erythrocyte membranes and their recruitment into the malarial vacuole (Lauer, S. A., VanWye, J., Harrison, T., McManus, H., Samuel, B. U., Hiller, N., Haldar, K. (2000). EMBO Journal. 19:1-9; Samuel, B. U., Narla, M., Harrison, T., Reid, M., Rosse, W., Haldar, K. (2001). J. Biol. Chem. 276:29319-29329; Harrison, T., Samuel, B. U., Akompong, T., Hamm, H. E., Narla, M., Lomasney, J., Haldar, K. (2003). Science. 301:1734-1736.; Hiller, N., Akompong, T., Morrow, J., Holder, A., Haldar, K. (2003). J. Biol. Chem. 278(48):48413-21.; Murphy, S., Samuel, B. U., Harrison, T., Speicher, K. D., Speicher, D. W., Reid, M., Prohaska, R., Low P. S., Tanner, M. J., Mohandas, N., Haldar, K. (2003). Blood, in press. The observation was made that while erythrocyte Gs, Gq and Gi all reside in rafts, only Gs is recruited to the malarial vacuole. In addition, the Gs receptor β2-adrenergic receptor (β2-AR) is also recruited, and studies were done to determine whether selective recruitment of Gs and its receptor had any functional implications. A dominant negative peptide approach was utilized. The dominant negative strategy taken utilized peptides that mimic the last 11 amino acids of the C terminus of G proteins; these peptides interfere with the ability of G-proteins to couple to G-protein coupled receptors. When these peptides were introduced into P. falciparumcultures, the peptide to the C terminus of Gαs specifically blocked erythrocytic infection during parasite entry, while scrambled peptides and those to Gq and Gi had no effect. Since the parasite genome fails to encode for heterotrimeric G proteins, the Gαs peptide selectively disrupts host Gαs-receptor interactions. Further, agonists to the Gs coupled receptors β-AR and the adenosine receptors that signal via cAMP stimulate infection. Antagonists (β-blockers such as racemic propranolol) inhibit agonist-stimulated infection of P. falciparum in vitro and also block P. berghei infections in mice. In contrast the inactive isomer of propranolol has no effect on either P. falciparum growth in culture or P. berghei infection in mice. On the basis of these data and since β-blockers are bioavailable, can be given orally, are well tolerated and inexpensive, they would make excellent candidates for development into new antimalarials.

### Screening Methods

Furthermore, another aspect of the invention provides for screening for compounds that down regulate G protein mediated functions in the host cell and development of these new anti-malarial compounds based on the novel strategy described herein for malarial chemotherapy. This invention further provides novel agents identified by the above-described screening assays and uses thereof for treatments as described herein. In particular, a further embodiment contemplates identifying new compounds through the screening methods described herein for use in treating mammals, including humans, that are suffering from malaria or the sequelae of malarial infection, or preventing malaria, or ameliorating the symptoms of malarial infection.

In another embodiment, agents that modulate (up-regulate or down-regulate) the expression or activity of G protein receptors are identified by contacting a preparation containing the cells bearing these receptors with a test compound or a control compound and determining the ability of the test compound to modulate (e.g., stimulate or inhibit) the expression or activity of the receptor. The expression or activity of the G protein receptor(s), including beta-adrenergic or adenosine receptors can be assessed in a number of ways, known to those skilled in the art, for example, methods found in U.S. Pat. Nos. 6,280,934; 6,291,177; 5,891,646; 6,383,761; 6,403,305, 5,783,402, incorporated herein by reference in their entireties. This invention further provides novel agents identified by the above-described screening assays and uses thereof for treatments as described herein.

For example, in a preferred embodiment, the method of identifying an antagonist of a G-protein coupled receptor that modulates adenylyl cyclase comprises:

a) transfecting a cell with the cDNA encoding the G-protein coupled receptor, wherein said cell also contains a cyclic AMP sensitive reporter construct;

b) expressing the G-protein coupled receptor;

c) adding a cyclic AMP inducer or a β-adrenergic agonist to the cell to induce production of cyclic AMP, and adding a test antagonist to said cell; and

d) determining whether the test antagonist binds to the receptor by determining whether the amount of cyclic AMP is inhibited or induced compared to the test cell to which the cyclic AMP inducer or β-adrenergic agonist, but not the test antagonist, has been added.

This method further comprises adding a chromogenic substrate and determining a change in the chromogenic substrate which indicates whether the amount of cyclic AMP is inhibited or induced. The chromogenic substrate may be o-nitrophenyl β-D-galactopyranoside. The method further comprises a G-protein coupled receptor whose subtype is a Gαₛ-protein coupled receptor and the determining step comprises determining whether the amount of cyclic AMP is inhibited compared to the test cell to which the cyclic AMP inducer but not the test antagonist has been added. The method further comprises expressing the G-protein coupled receptor in the presence of an agonist of the Gαₛ-protein coupled receptor, and determining whether the test antagonist binds to the receptor and determining whether the amount of cyclic AMP in the test cell to which both the agonist and the test antagonist have been added is increased compared to the test cell to which the known agonist but not the antagonist have been added. For example, such analyses can be performed using the following protocol: erythrocyte membranes or whole cells are stimulated with agonists such as adrenaline, isoproterenol, NECA (at concentrations ranging from 10⁻¹² M to 10⁻⁵ M) in the presence or absence of the peptide or antagonist of interest. Membrane incubations require addition of GTP, ATP, Mg⁺², Mn⁺² and Ca⁺² (each ion used at 10 mM). Cyclic AMP can be measured in erythrocytes using a Direct Cyclic AMP Enzyme Immunoassay kit (Assay Designs Inc.) according to the manufactures instructions. Control experiments are carried out in the presence of an antagonist like propanolol (which is known to affect isoproterenol mediated activation of cAMP in red cells) or in the absence of either agonist or antagonist. It is also possible to measure signaling downstream of cAMP activation. For example, PKA activation is measured by ³²Pi labeling of cellular proteins in incubations containing, agonist, antagonist, both or neither (but contains solvent used for ligand delivery), and can be detected by SDS-PAGE and fluorography. The effect of Gs and G scrambled peptide on specific activation of cAMP and PKA dependent phosphorylation also can be examined.

In another preferred embodiment, a high throughput screening method is used to identify novel antagonists. This method incorporates use of a GFP tagged P. berghei parasite line to carry out assays by flow cytometry rather than Giemsa staining. In vivo mouse experiments are performed with a cytosolic-GFP tagged strain of P. berghei using a standard 4 day Peters assay (W. Peters, B. L. Robinson, Ann Trop Med Parasitol 78, 561 (1984)). P. berghei and the novel antagonists are administered intraperitoneally; five mice are used per data point. Mice are given 5×10⁷ parasites on day 0, and doses of novel drug are given twice a day on days 0-3. Tail bleeds are carried out on day 4 to ascertain parasitemia in the presence and absence of antagonist. When collecting blood from tail bleed, one drop of blood from the animal is diluted in 1 ml of PBS. This tissue is fixed with 1% formaldehyde in PBS. After fixation, the cells are washed three times with 1 ml PBS. The number of green (GFP) events in 10,000 cells is counted using flow cytometry. A decrease in the number of green events in 10,000 cells indicates the effectiveness of antagonist as an anti-malarial compound.

In another embodiment, agents that modulate (i.e., up-regulate or down-regulate) the expression or activity of G protein receptors, including but not limited to, beta adrenergic and adenosine receptors, are identified in an animal model. Examples of suitable animals include, but are not limited to, mice, rats, and monkeys. Preferably, the animal used represents a model of malarial infection. Such models of malaria infection can be obtained as known in the art. For example, mouse and rat models of malaria infection can be obtained by injecting sporozites via the tail vein at a concentration of 2×10⁶ sporozoites per animal (Lau A O, Sacci J B Jr, Azad A F. J Immunol. 2001 Feb. 1;166(3):1945-50) Primate models of malaria can be obtained by methods known in the art as well. For example, Aotus monkeys can be infected using the FVO strain of P. falciparum adapted to Aotus monkeys. The monkeys can be inoculated intravenously with up to 500,000 parasitized red blood cells (pRBC) obtained from a donor Aotus monkey and evaluated for the symptoms of malaria (Carvalho, L; Alves, F A; Oliveira, S G et al. Mem. Inst. Oswaldo Cruz, July 2003, vol.98, no.5, p.679-686.; Canfield C J, Milhous W K, Ager A L, Rossan R N, Sweeney T R, Lewis N J, Jacobus D P. Am J Trop Med Hyg. 1993 July;49(1):121-6.)

### Pharmaceutical Compositions

As demonstrated by the methods of the present invention, although G proteins are coupled to receptors and mediate a number of signaling events in a wide variety of cells, their function in mature red cells has been poorly studied. Furthermore, since the red cell is e-nucleated, has no intracellular structures, and is incapable of de novo protein and lipid biosynthesis, the requirement for G protein function in this cell remains unclear. It has been established that erythrocyte Gs is recruited to the malarial vacuole when red cells are infected with the parasite (Lauer et al., 2000). One aspect of the current invention provides for testing whether the recruitment of erythrocyte Gs to the malarial vacuole following infection had functional consequences. In one specific embodiment, peptides derived from the C terminal region of Gs that block the interaction of Gs with its receptors were introduced and tested for their effects on malaria infection in in vitro culture. These studies demonstrated that Gs peptides gained access to the red cell as the parasite entered the cell and blocked the establishment of intracellular infection.

The only known Gs coupled receptors on the red cell are the beta-adrenergic and the adenosine receptors (Horga J F, Gisbert J, De Agustin J C, Hernandez M, Zapater P; Blood Cells Mol Dis. (2000) Jun. 26(3):223-8; Tuvia S, Moses A, Gulayev N, Levin S, Korenstein R.; J Physiol. (1999) May 1; 516 (Pt 3):781-92; Mazzoni M R, Taddei S, Giusti L, Rovero P, Galoppini C, D'Ursi A, Albrizio S, Triolo A, Novellino E, Greco G, Lucacchini A, Hamm H E (2000) Mol Pharmacol. 58(1):226-36). In a further aspect of the invention, the role of G protein mediated function in malarial infection was provided for by elucidating the effects of agonists and antagonists of the beta-adrenergic and adenosine receptors on the establishment of infection with the malarial parasite. The results of the studies provided herein demonstrate that agonists of both receptors stimulate parasite infection of the red cell.

Accordingly, in a more specific embodiment, pharmaceutical compositions comprising antagonists to the beta-adrenergic and adenosine receptors are contemplated for methods of treating a mammal suffering from malaria or the sequelae of malaria, or for methods for preventing malaria, or for ameliorating the symptoms of malarial infection combined with a pharmaceutically acceptable carrier. In a further embodiment, a combination of antagonists is envisioned since such a combination inhibits parasite growth in a rodent model of malaria.

Experimental evidence provided herein demonstrates that interfering with Gs-receptor interactions blocks malarial infection. None of the presently available anti-malarial compounds are targeted against G proteins. Thus, the studies provided in the current application indicate that inhibition of G protein function (including the use of beta-blockers that down regulate Gs receptors) provides a new chemotherapeutic approach to cure malaria and to treat malarial parasites resistant to existing drugs.

Thus, a further embodiment contemplates identifying new compounds through the screening methods described herein for use in treating mammals, including humans, that are suffering from malaria or the sequelae of malarial infection, or preventing malaria, or ameliorating the symptoms of malarial infection. A further aspect of the invention is the use of other known compounds that down regulate G protein mediated functions, for use in treating mammals, including humans, that are suffering from malaria or the sequelae of malarial infection, or for preventing malaria, or for ameliorating the symptoms of malarial infection.

The pharmaceutical compositions comprising antagonists to the beta-adrenergic and adenosine receptors are formulated to contain a therapeutically effective amount of the antagonists described herein and a pharmaceutically acceptable carrier. In a particular embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents.

The therapeutic agent, whether it be a polypeptide, analog or active fragment-containing compositions or small organic molecules, are conventionally administered by various routes including intravenously, intramuscularly, subcutaneously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of inhibition or neutralization of binding capacity desired. Precise amounts of active ingredient required to be administered depend on the judgment of the practitioner and are peculiar to each individual. Suitable regimes for initial administration and subsequent injections are also variable, but are typified by an initial administration followed by repeated doses at intervals by a subsequent injection or other administration.

These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the subject. The formulation should suit the mode of administration.

The compounds of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

Administration of the compositions to the site of injury, the target cells, tissues, or organs, may be by way of oral administration as a pill or capsule or a liquid formulation or suspension. It may be administered via the transmucosal, sublingual, nasal, rectal or transdermal route. Parenteral administration may also be via intravenous injection, or intramuscular, intradermal or subcutaneous. Due to the nature of the diseases or conditions for which the present invention is being considered, the route of administration may also involve delivery via suppositories. This is especially true in conditions whereby the ability of the patient to swallow is compromised.

The plant compositions or extracts may be provided as a liposome formulation. Liposome delivery has been utilized as a pharmaceutical delivery system for other compounds for a variety of applications. See, for example Langer (1990) Science 249:1527-1533; Treat et al. (1989) in Liposomes in the Therapy of Infectious Disease and Cancer, Lopez-Berestein and Fidler (eds.), Liss: New York, pp. 353-365 (1989). Many suitable liposome formulations are known to the skilled artisan, and may be employed for the purposes of the present invention. For example, see: U.S. Pat. No. 5,190,762.

In a further aspect, liposomes can cross the blood-brain barrier, which would allow for intravenous or oral administration. Many strategies are available for crossing the blood-brain barrier, including but not limited to, increasing the hydrophobic nature of a molecule; introducing the molecule as a conjugate to a carrier, such as transferrin, targeted to a receptor in the blood-brain barrier; and the like.

Transdermal delivery of the compositions is also contemplated. Various and numerous methods are known in the art for transdermal administration of a drug, e.g., via a transdermal patch. It can be readily appreciated that a transdermal route of administration may be enhanced by use of a dermal penetration enhancer.

Controlled release oral formulations may be desirable. The composition may be incorporated into an inert matrix which permits release by either diffusion or leaching mechanisms, e.g., gums. Slowly degenerating matrices may also be incorporated into the formulation. Some enteric coatings also have a delayed release effect. Another form of a controlled release of this therapeutic is by a method based on the Oros therapeutic system (Alza Corp.), i.e. the drug is enclosed in a semipermeable membrane which allows water to enter and push drug out through a single small opening due to osmotic effects.

Pulmonary delivery may be used for treatment as well. Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. With regard to construction of the delivery device, any form of aerosolization known in the art, including but not limited to spray bottles, nebulization, atomization or pump aerosolization of a liquid formulation, and aerosolization of a dry powder formulation, can be used in the practice of the invention.

Ophthalmic and nasal delivery may be used in the method of the invention. Nasal delivery allows the passage of a pharmaceutical composition of the present invention to the blood stream directly after administering the therapeutic product to the nose, without the necessity for deposition of the product in the lung. Formulations for nasal delivery include those with dextran or cyclodextrins. For nasal administration, a useful device is a small, hard bottle to which a metered dose sprayer is attached. In one embodiment, the metered dose is delivered by drawing the pharmaceutical composition of the present invention solution into a chamber of defined volume, which chamber has an aperture dimensioned to aerosolize and aerosol formulation by forming a spray when a liquid in the chamber is compressed. The chamber is compressed to administer the pharmaceutical composition of the present invention. In a specific embodiment, the chamber is a piston arrangement. Such devices are commercially available.

The compositions of the present invention are also suited for transmucosal delivery. In particular, the compositions and extracts are particularly suited for sublingual, buccal or rectal delivery of agents that are sensitive to degradation by proteases present in gastric or other bodily fluids having enhanced enzymatic activity. Moreover, transmucosal delivery systems can be used for agents that have low oral bioavailability. The compositions of the instant invention comprise the compound dissolved or dispersed in a carrier that comprises a solvent, an optional hydrogel, and an agent that enhances transport across the mucosal membrane. The solvent may be a non-toxic alcohol known in the art as being useful in such formulations of the present invention and may include, but not be limited to ethanol, isopropanol, stearyl alcohol, propylene glycol, polyethylene glycol, and other solvents having similar dissolution characteristics. Other such solvents known in the art can be found in “The Handbook of Pharmaceutical Excipients”, published by The American Pharmaceutical Association and The Pharmaceutical Society of Great Britain (1986) and the Handbook of Water-Soluble Gums and Resins, ed. By R. L. Davidson, McGraw-Hill Book Co., New York, N.Y. (1980).

Any transmucosal preparation suitable for administering the components of the present invention or a pharmaceutically acceptable salt thereof can be used. Particularly, the mixture is any preparation usable in oral, nasal, or rectal cavities that can be formulated using conventional techniques well known in the art. Preferred preparations are those usable in oral, nasal or rectal cavities. For example, the preparation can be a buccal tablet, a sublingual tablet, and the like preparation that dissolve or disintegrate, delivering drug into the mouth of the patient. A spray or drops can be used to deliver the drug to the nasal cavity. A suppository can be used to deliver the mixture to the rectal mucosa. The preparation may or may not deliver the drug in a sustained release fashion.

A specific embodiment for delivery of the components of the present invention is a mucoadhesive preparation. A mucoadhesive preparation is a preparation which upon contact with intact mucous membrane adheres to said mucous membrane for a sufficient time period to induce the desired therapeutic or nutritional effect. The preparation can be a semisolid composition as described for example, in WO 96/09829. It can be a tablet, a powder, a gel or film comprising a mucoadhesive matrix as described for example, in WO 96/30013. The mixture can be prepared as a syrup that adheres to the mucous membrane.

Suitable mucoadhesives include those well known in the art such as polyacrylic acids, preferably having the molecular weight between from about 450,000 to about 4,000,000, for example, Carbopol™934P; sodium carboxymethylcellulose (NaCMC), hydroxypropylmethylcellulose (HPMC), or for example, Methocel™ K100, and hydroxypropylcellulose.

The delivery of the components of the present invention can also be accomplished using a bandage, patch, device and any similar device that contains the components of the present invention and adheres to a mucosal surface. Suitable transmucosal patches are described for example in WO 93/23011, and in U.S. Pat. No. 5,122,127, both of which are hereby incorporated by reference. The patch is designed to deliver the mixture in proportion to the size of the drug/mucosa interface. Accordingly, delivery rates can be adjusted by altering the size of the contact area. The patch that may be best suited for delivery of the components of the present invention may comprise a backing, such backing acting as a barrier for loss of the components of the present invention from the patch. The backing can be any of the conventional materials used in such patches including, but not limited to, polyethylene, ethyl-vinyl acetate copolymer, polyurethane and the like. In a patch that is made of a matrix that is not itself a mucoadhesive, the matrix containing the components of the present invention can be coupled with a mucoadhesive component (such as a mucoadhesive described above) so that the patch may be retained on the mucosal surface. Such patches can be prepared by methods well known to those skilled in the art.

Preparations usable according to the invention can contain other ingredients, such as fillers, lubricants, disintegrants, solubilizing vehicles, flavors, dyes and the like. It may be desirable in some instances to incorporate a mucous membrane penetration enhancer into the preparation. Suitable penetration enhancers include anionic surfactants (e.g. sodium lauryl sulphate, sodium dodecyl sulphate), cationic surfactants (e.g. palmitoyl DL camitine chloride, cetylpyridinium chloride), nonionic surfactants (e.g. polysorbate 80, polyoxyethylene 9-lauryl ether, glyceryl monolaurate, polyoxyalkylenes, polyoxyethylene 20 cetyl ether), lipids (e.g. oleic acid), bile salts (e.g. sodium glycocholate, sodium taurocholate), and related compounds.

The administration of the compositions and extracts of the present invention can be alone, or in combination with other compounds effective at treating the various medical conditions contemplated by the present invention. Also, the compositions and formulations of the present invention, may be administered with a variety of analgesics, anesthetics, or anxiolytics to increase patient comfort during treatment.

The compositions of the invention described herein may be in the form of a liquid. The liquid may be delivered as a spray, a paste, a gel, or a liquid drop. The desired consistency is achieved by adding in one or more hydrogels, substances that absorb water to create materials with various viscosities. Hydrogels that are suitable for use are well known in the art. See, for example, Handbook of Pharmaceutical Excipients, published by The American Pharmaceutical Association and The Pharmaceutical Society of Great Britain (1986) and the Handbook of Water-Soluble Gums and Resins, ed. By R. L. Davidson, McGraw-Hill Book Co., New York, N.Y. (1980).

Suitable hydrogels for use in the compositions include, but are not limited to, hydroxypropyl cellulose, hydroxypropyl methyl cellulose, sodium carboxymethyl cellulose and polyacrylic acid. Preferred hydrogels are cellulose ethers such as hydroxyalkylcellulose. The concentration of the hydroxycellulose used in the composition is dependent upon the particular viscosity grade used and the viscosity desired in the final product. Numerous other hydrogels are known in the art and the skilled artisan could easily ascertain the most appropriate hydrogel suitable for use in the instant invention.

The mucosal transport enhancing agents useful with the present invention facilitate the transport of the agents in the claimed invention across the mucosal membrane and into the blood stream of the patient. The mucosal transport enhancing agents are also known in the art, as noted in U.S. Pat. No. 5,284,657, incorporated herein by reference. These agents may be selected from the group of essential or volatile oils, or from non-toxic, pharmaceutically acceptable inorganic and organic acids. The essential or volatile oils may include peppermint oil, spearmint oil, menthol, eucalyptus oil, cinnamon oil, ginger oil, fennel oil, dill oil, and the like. The suitable inorganic or organic acids useful for the instant invention include but are not limited to hydrochloric acid, phosphoric acid, aromatic and aliphatic monocarboxylic or dicarboxylic acids such as acetic acid, citric acid, lactic acid, oleic acid, linoleic acid, palmitic acid, benzoic acid, salicylic acid, and other acids having similar characteristics. The term “aromatic” acid means any acid having a 6-membered ring system characteristic of benzene, whereas the term “aliphatic” acid refers to any acid having a straight chain or branched chain saturated or unsaturated hydrocarbon backbone.

Other suitable transport enhancers include anionic surfactants (e.g. sodium lauryl sulphate, sodium dodecyl sulphate), cationic surfactants (e.g. palmitoyl DL camitine chloride, cetylpyridinium chloride), nonionic surfactants (e.g. polysorbate 80, polyoxyethylene 9-lauryl ether, glyceryl monolaurate, polyoxyalkylenes, polyoxyethylene 20 cetyl ether), lipids (e.g. oleic acid), bile salts (e.g. sodium glycochoiate, sodium taurocholate), and related compounds.

When the compositions and extracts of the instant invention are to be administered to the oral mucosa, the preferred pH should be in the range of pH 3 to about pH 7, with any necessary adjustments made using pharmaceutically acceptable, non-toxic buffer systems generally known in the art.

For topical delivery, a solution of the compound in water, buffered aqueous solution or other pharmaceutically-acceptable carrier, or in a hydrogel lotion or cream, comprising an emulsion of an aqueous and hydrophobic phase, at a concentration of between 50 μM and 5 mM, is used. A preferred concentration is about 1 mM. To this may be added ascorbic acid or its salts, or other ingredients, or a combination of these, to make a cosmetically-acceptable formulation. Metals should be kept to a minimum. It may be preferably formulated by encapsulation into a liposome for oral, parenteral, or, preferably, topical administration.

The invention provides methods of treatment comprising administering to a subject a therapeutically effective amount of at least one G protein coupled receptor antagonist. In one embodiment, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human. In one specific embodiment, a non-human mammal is the subject. In another specific embodiment, a human mammal is the subject.

The amount of beta adrenergic or adenosine receptor antagonist which is optimal in treating malaria can be determined by standard clinical techniques based on the present description. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each subject's circumstances. However, suitable dosage ranges for intravenous administration are generally about 20-500 micrograms of active compound per kilogram body weight. Suitable dosage ranges for i.p. delivery are expected to lie between 7-100 mg/kg. Suitable dosage ranges for intranasal administration are generally about 0.01 pg/kg body weight to 1 mg/kg body weight. Effective doses may be extrapolated from dose-response curves derived from in vitro or animal model test systems.

### EXAMPLES

### Example 1

### Effect of Blocking the Interaction of Gs with its Receptor on Malarial Infection

In a specific embodiment, studies were done to investigate whether recruitment of Gs to the malarial vacuole had functional consequences for infection. Thus, experiments were done to determine whether blocking the interaction of Gs with its receptor(s) in the red cell membrane could influence malarial infection. A dominant negative strategy (Gilchrist et al., 1999; Gilchrist et al., 2001) was employed for studying G proteins. In G proteins GTP is complexed with Mg2+ and GTP and magnesium binding sites are tightly coupled. Current dominant-negative strategies target Mg2+ binding sites but tend to be leaky. In the alpha subunit, the best characterized receptor contact is at the C terminus, of which the last 7 amino acids are the most important. Hamm and colleagues have shown that Gα C terminal peptides can competitively block G protein-coupled downstream events. Thus, the effects of introducing C terminal peptides for Gs on malarial infection were studied. These peptides are acetylated to facilitate their delivery into cells.

As shown in Tables I and II, when added at extracellular concentrations of 100-500 μM, the Gαs peptide (QRMHLRQYELL (SEQ ID NO:1)) appeared to inhibit parasite growth in culture, in a dose dependent manner, by blocking the establishment of new ring stage parasites. Control peptides balanced for overall charge and mass such as the scrambled Gs peptide Gscb1 (ELRLQHYMQLR (SEQ ID NO:2)) at 500 μM had virtually no effect. Gαq and Gαi are heterotrimeric G proteins of the red cell that are not recruited to the plasmodial vacuole. Consistently, the C terminal peptide of Gq (LQLNLKEYNLV (SEQ ID NO: 3)) and the reverse of C terminal peptide Gri (NGIKCLFNDKL (SEQ ID NO:4)) had little effect on plasmodial growth. Taken together the data suggest that the effect of the Gs peptide may be specific and due to selective action against Gαs receptor function during malarial infection.

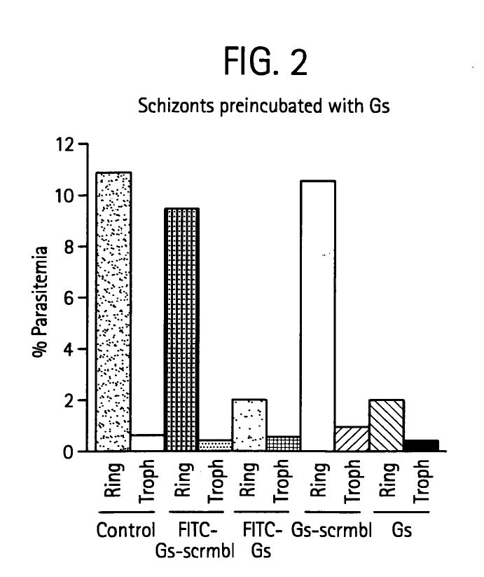

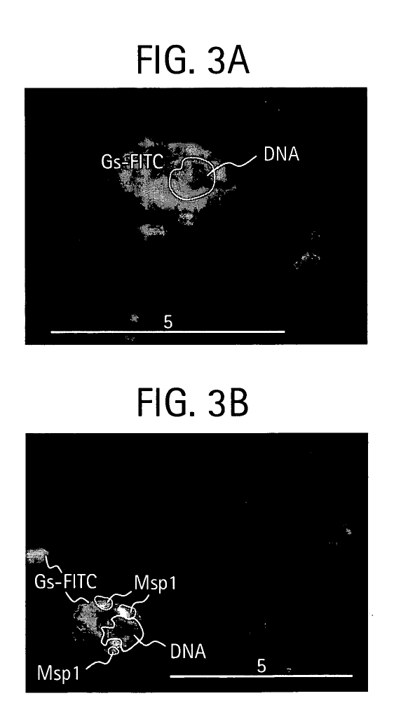

To examine this further, purified schizonts were incubated with uninfected red cells and the cultures were scored for new ring formation as described. As shown, Gs specifically induced a marked inhibition of new rings (FIG. 2). N-terminal FITC-derived forms of both peptides can be seen in association of newly formed rings (FIG. 3), suggesting that both are delivered to the site of infection. In infected cells fluorescent peptide is detected in association with the parasite and red cell membrane (see FIG. 3).

A recent report in the literature suggests that Plasmodium merozoites in schizonts contain heterotrimeric G proteins (Dyer and Day, 2000). These parasite G proteins have been proposed to function in gametocytogenesis and not asexual development. Terminal schizonts/merozoites were pre-incubated with the Gs peptide and it was determined that this had no detrimental effect on infection (not shown). Thus it appears that the preferred site of Gs peptide action is not in competition with a parasite encoded Gs. Data base searches fail to reveal the presence of a plasmodial homologue of Gs or any malarial proteins that contain the indicated Gs peptide sequence. It has been suggested that divergent plasmodial G proteins may exist but the action of the peptide is based on sequence based inhibition of conserved interactions. This does not rule out the presence of divergent malarial Gs, but we are quite confident that the effects of the Gas peptide in our studies does not result from its competitive effects with a divergent protein but rather than the host Gs molecule.

Since the Gαs peptide is expected to act by competitive inhibition it should induce a loss in Gs signaling. Gαs is known to couple receptors to the stimulation of adenylate cyclase (Skiba and Hamm, 1998). Red blood cells are known to contain two Gs coupled receptors the beta-adrenergic receptor (βAR) and adenosine receptor (Horga J F, Gisbert J, De Agustin J C, Hernandez M, Zapater P Blood Cells Mol Dis.(2000) Jun.;26(3):223-8; Tuvia S, Moses A, Gulayev N, Levin S, Korenstein R. J Physiol. (1999) May 1;516 (Pt 3):781-92.; Mazzoni M R, Taddei S, Giusti L, Rovero P, Galoppini C, D'Ursi A, Albrizio S, Triolo A, Novellino E, Greco G, Lucacchini A, Hamm H E (2000). Mol Pharmacol. 58(1):226-36). Thus the effects of Gαs and control peptides on βAR and adenosine stimulated red blood cells was examined. It is believed that the Gs peptide acts to block malarial invasion but does not interfere with the initial interaction of the parasite with the receptor on the red cell. A combination of fluorescence and electron microscopy is used to define the association of the parasites with red cells in the presence and absence of control and inhibitory peptides.

### Example 2

### Methods for Testing the Effect of Agonists and Antagonists of G Protein Receptors on Malarial Infection

To examine the effect of antagonists of beta adrenergic receptor (βAR) or adenosine receptor on malarial invasion, compounds such as propranolol (10⁻⁸ to 10⁻⁵ M), known to antagonize the βAR, or 8-SPT (10⁻⁸ to 10⁻⁵ M), known to antagonize the adenosine receptor, were tested for their effects on new ring formation. These studies have established that antagonists to these receptors can block cAMP activation and inhibit infection in red blood cells.

### Protocol

Infection assays were set up using synchronized cultures of P. falciparum with a starting parasitemia of 6% schizonts. Cultures were incubated with 500 uM of the indicated peptides (Gs, Gs-scrambled, Gq or Gi-reversed) or mock treated (with no peptide) overnight and subsequently scored for new ring stage infection by Giemsa staining of thin blood smears. Infection is shown compared to that of mock treated cultures (containing no peptide). (Table 1; Standard error: 10%.)

### Results

As shown in Table I below, a peptide from the C-terminal end of the heterotrimeric G protein Gs, when added to cultures of P. falciparum, significantly reduced erythrocytic infection. This peptide is known to block interactions between Gs proteins and their component receptors. In contrast, the peptide Gs-scrambled that contains the same amino acids but in a different sequence does not block infection. Peptides related to Gi and Gq also show no significant effect on infection. These results suggest that effect of the Gs peptide on infection is dependent on its sequence, and further suggest that blocking Gs function is detrimental to infection. Data base searches fail to detect Gs sequences in the P. falciparum genome: the genome has now been sequenced and there is no evidence for a parasite encoded heterotrimeric G protein. Hence the Gs peptides used in the present invention are believed to disrupt host G protein—receptor interactions.

TABLE I

Effects of C terminal peptides of erythrocyte
heterotrimeric G proteins on malarial
infection.

| | | | |
|---|---|---|---|
| | % Inhibition of | | |
| Peptide | Infection | Peptide | Sequence |

| | | | |
|---|---|---|---|
| Gs | 87 | Gs | QRMHLRQYELL |
| Gs-scrambled | 4 | Gri | NGIKCLFNDKL |
| Gq | 20 | Gq | LQLNLKEYNLV |
| Gi-reverse | 6 | | |

Table II illustrates the dose dependent effect of Gs peptide on inhibition of P. falciparum infection. In this study, ring-infected cultures at 1% parasitemia were incubated with the indicated concentrations of Gs peptides under standard culture conditions and monitored for development to the subsequent trophozoite stage as well as the next generation of ring stage parasites. Parasitemias were detected by Giemsa staining of thin blood smears. Standard error: 10%.

As shown in Table II, Gs peptide has a marked effect on new ring formation, suggesting that it may influence the establishment of infection. The extracellular concentrations required for significant inhibition are in the high micromolar range (>50 μM) and this led us to further investigate how the peptide might be delivered to the site of infection.

TABLE II
Dose Dependent Inhibition with Gs Peptide

| | | μM Gs Peptide | | | | | | |
|---|---|---|---|---|---|---|---|---|
| | | 0 | 5 | 20 | 50 | 100 | 200 | 500 |
| Day 1 | % Ring | 1.0 | 1.0 | 1.0 | 1.0 | 1.0 | 1.0 | 1.0 |
| | % Troph | — | — | — | — | — | — | — |
| Day 2 | % Ring | — | — | — | — | — | — | — |
| | % Troph | 0.8 | 0.9 | 1.0 | 0.9 | 0.8 | 1.0 | 0.9 |
| Day 3 | % Ring | 5.2 | 3.8 | 2.7 | 1.9 | 1.4 | 0.6 | 0.4 |
| | % Troph | 0.4 | 0.6 | 0.6 | 0.3 | 0.6 | 0.5 | 0.5 |

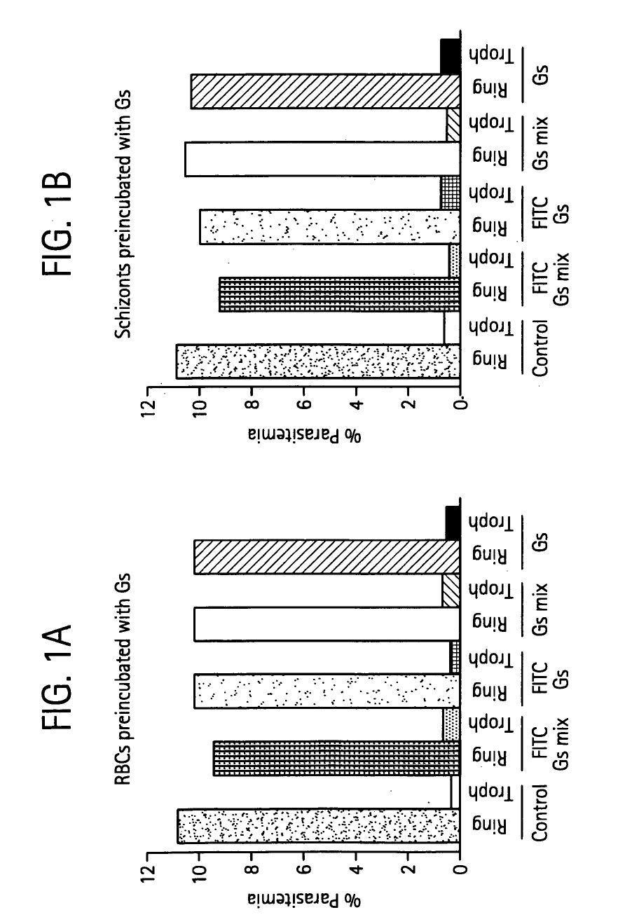

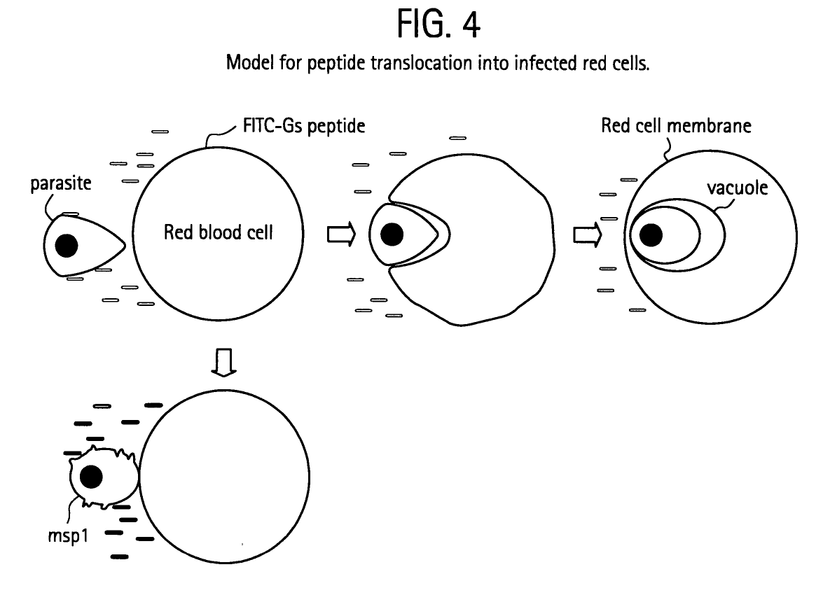

As shown in FIG. 1, pre-incubating either red cells or late stage segmenters for four hours with 500 μM Gs peptide failed to block infection, suggesting that (although this peptide is acetylated), it does not efficiently diffuse into cells. Studies using FITC-conjugated forms of Gs and Gs scrambled peptides (shown to display the activities associated with their non conjugated counterparts; see FIG. 2) suggested that peptides access red cells and the nascent or early ring vacuole at the time of parasite entry (FIG. 3A). Since invasion is a rapid process relatively small amounts of Gs peptide are likely to internalized (As summarized in FIG. 4), thereby explaining the need for high extracellular concentrations needed in Tables I and II.

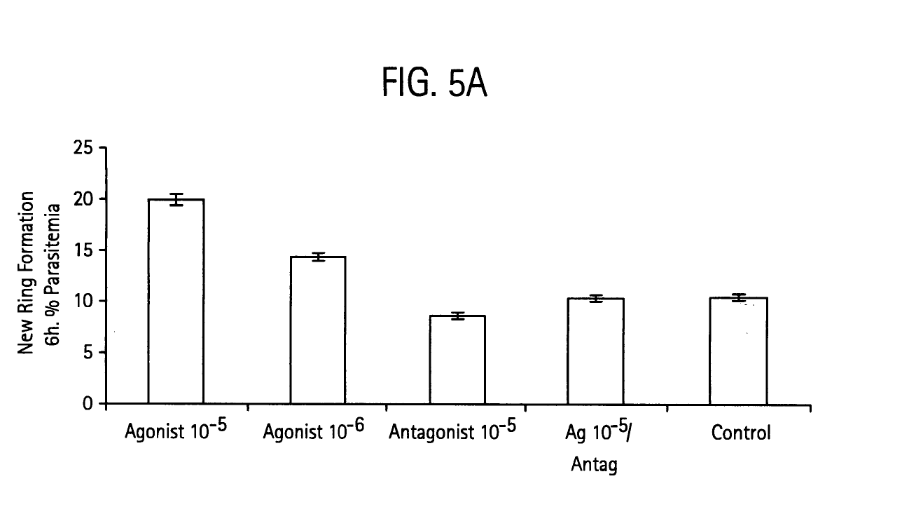

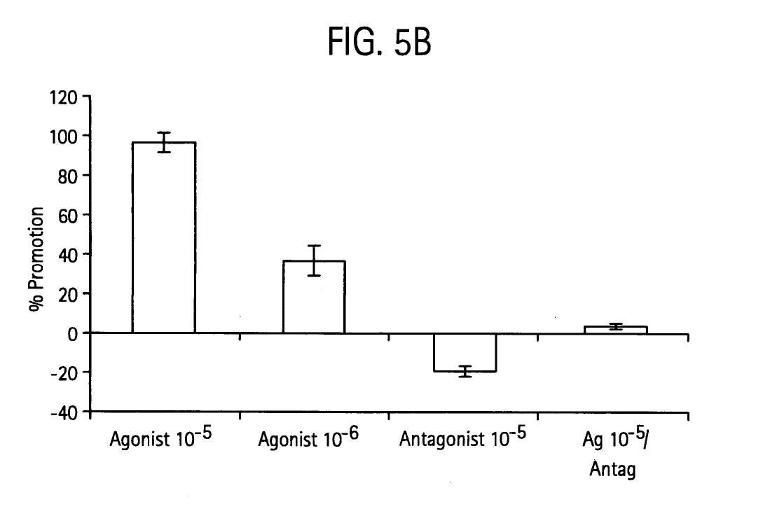

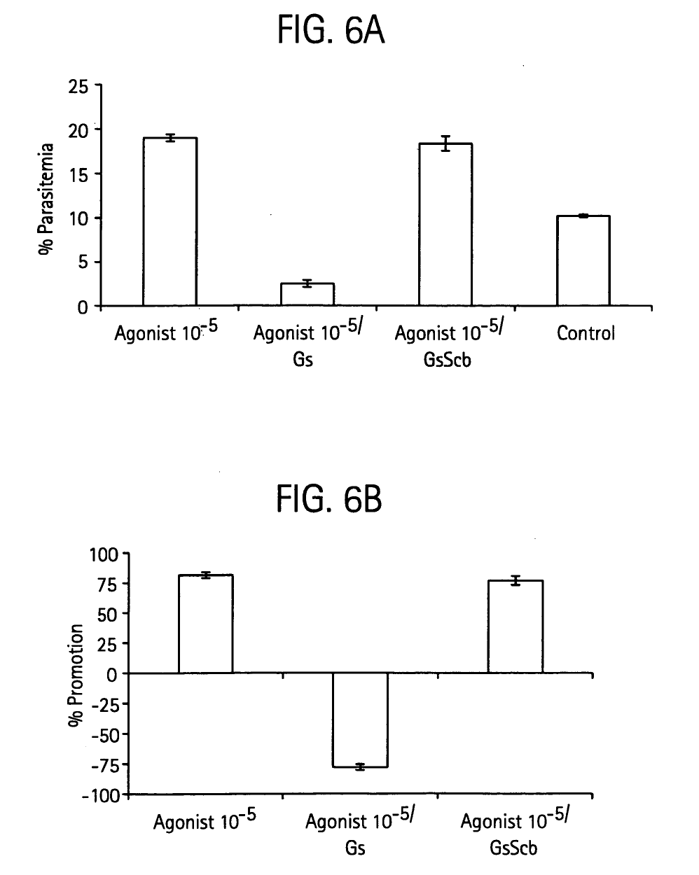

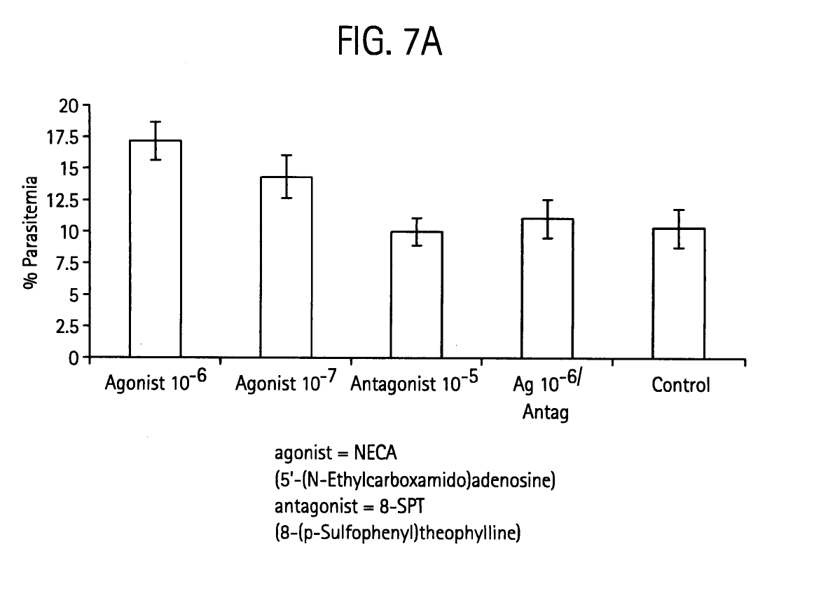

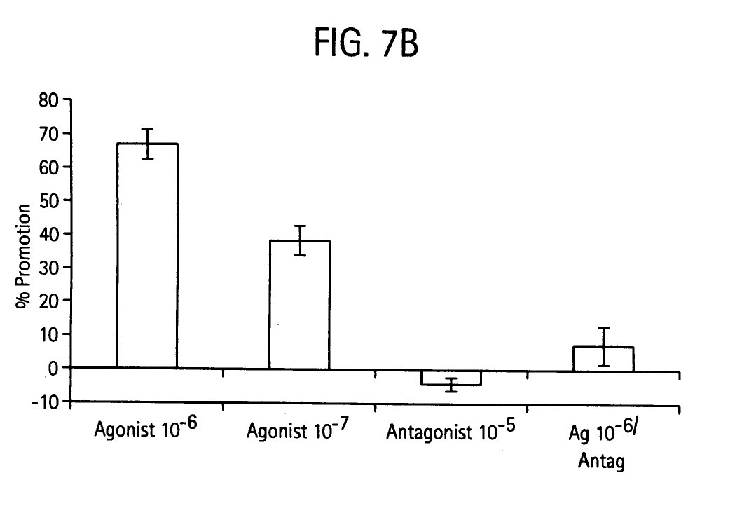

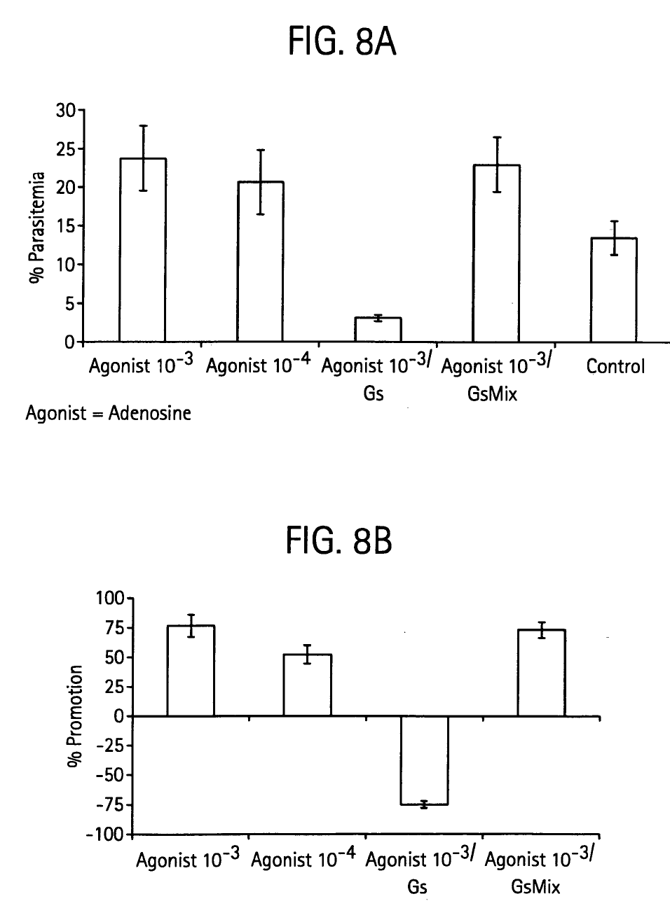

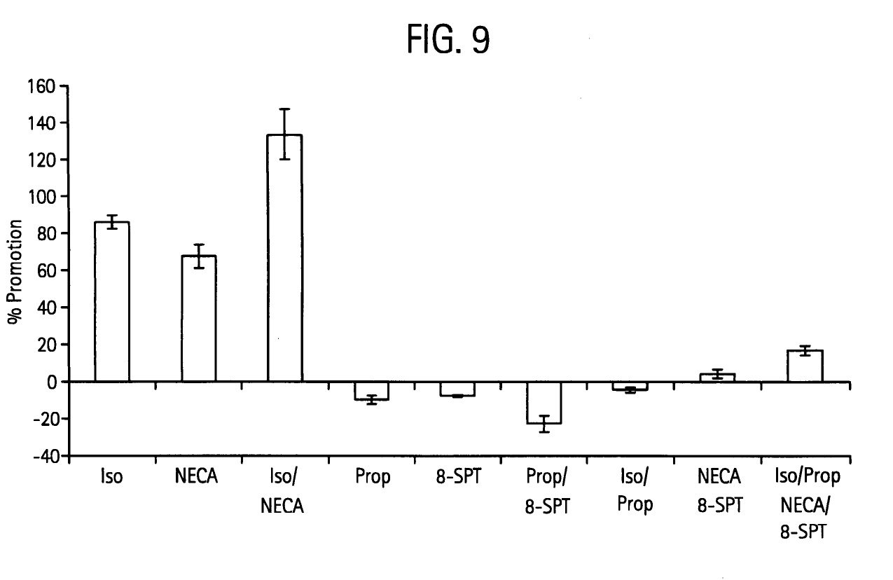

The β-adrenergic receptor and the adenosine receptor are two major Gs associated receptors known to be present on red cells. We were therefore interested in determining whether the inhibitory effects of the Gs peptides reflected a function of host Gs receptors in erythrocytic infection by P. falciparum. As shown in FIGS. 5-8, agonists of both the adenosine and the beta-adrenergic receptors stimulated infection of P. falciparum in vitro, while antagonists blocked this stimulation. A combination of antagonists were the most effective in decreasing infection (FIG. 9). This led us to investigate whether antagonist combinations could influence parasite proliferation in an animal model of malaria.

In a further study, animals were administered 10⁸ P. berghei parasites on day 0 intraperitoneally. The indicated amounts of antagonists or buffer alone (control) were administered twice daily from day 0 to day 4. Tail bleeds were performed on day 4 to determine parasitemia by Giemsa staining.

As shown below in Table III, a combination of the antagonists propranolol (at 3 mg/Kg) and 8-SPT (25 mg/Kg) administered twice daily reduced P. berghei infection by ˜40%. There was no change in weight, grooming of activity of animals treated with the drugs, suggesting that inhibition of Gs receptor function, and specifically antagonists of Gs receptors, may provide new approaches for treating malaria.

TABLE III
Effects of antagonists on plasmodial infection
in a P. berghei model of infection in rats.

| | Control | Drug Mix * |
|---|---|---|
| | n = 4 | n = 5 |
| % Parasitemia | 22.8 +/− 1.2 | 13.1 +/− 2.6 |
| % Inhibition | — | 42.5 |

As shown above in Table III, infected rats treated with the drug mix indicated above showed significant inhibition of parasitemia (42.5%).

### Example 3

### In vitro Effect of Various β-Adrenergic Antagonists on Inhibition of P. falciparum Infection in Red Blood Cells Treated with Isoproterenol

In vitro infection assays were done with synchronized cultures of P. falciparum (strain 3D7) using standard culture conditions. A starting parasitemia of 2.5% schizonts (44-48 h in development) in 20 μl of red blood cells with 1 ml of 10% human serum in RPMI1640 was used. The cultures were incubated for 4-6 hours in the presence of 10 μM isoproterenol or antagonist, or vehicle control. All detectable schizonts ruptured, and new ring stage infection was scored by Giemsa staining of thin blood smears. In all experiments, control cultures achieved ring parasitemias of 8-11%, and standard error was 10%.

### Results

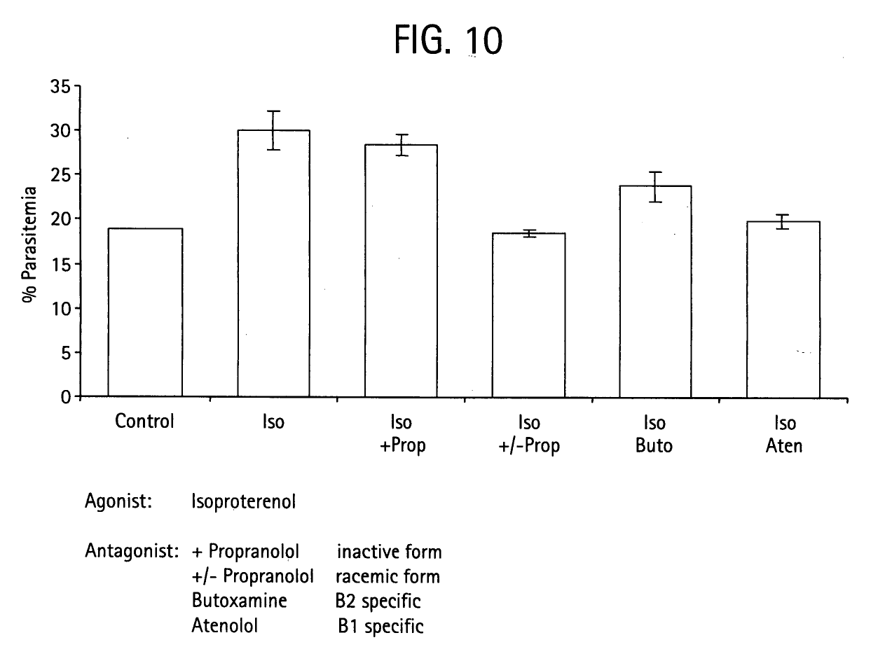

The In vitro studies suggest that beta 1 antagonists are likely to be more effective. β1 and β2 receptors are present on erythrocytes: isoproterenol and propranolol respectively activate and block both receptors. However as shown in FIG. 10, the β1 antagonist, altenolol is almost as effective as propranolol at blocking the stimulation of infection by P. falciparum in vitro. In contrast, butoxamine, a β2 specific antagonist shows a lower inhibitory effect.

### Example 4

### The Effect of the Racemic Form of Propranolol on Growth of P. falciparum in vivo

In vivo mouse experiments were done with P. berghei using a standard 4 day Peters assay (W. Peters, B. L. Robinson, Ann Trop Med Parasitol 78, 561 (1984)). P. berghei and β2-adrenergic receptor antagonists were administered intraperitoneally; five mice were used per data point. In IC₅₀ studies, 5 data points were taken. Mice were given 5×10⁷ parasites on day 0, and indicated concentrations of drug twice a day on days 0-3. Tail bleeds were carried out on day 4 to ascertain parasitemia, after which the animals were sacrificed.

### Results

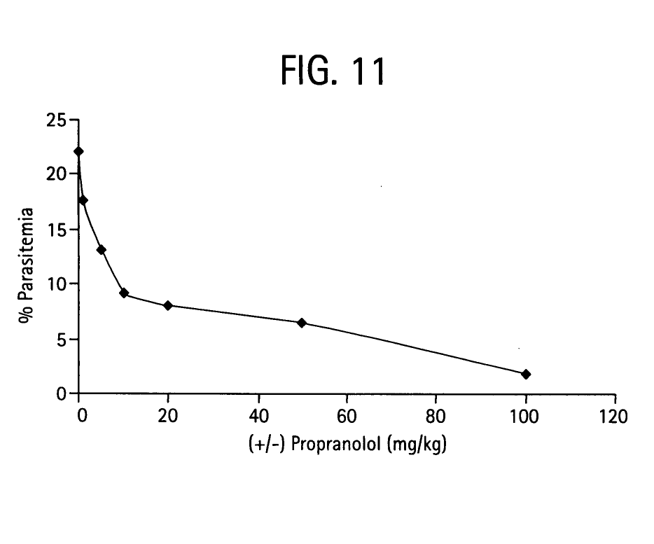

The IC50 and IC90 of racemic propranolol are 7.5 mg/Kg/day and 85 mg/Kg/day (drug is delivered intraperitoneally) for P. berghei infections in mice as shown in FIG. 11. The animals show no overt toxicity at either dose, as determined by weight, grooming, eyes and activity. At the higher dose, immediately after the injection there is reduction of motor activity proximal to the site of injection, but the effect is temporary. These data show that beta blockers alone (albeit at high concentrations) may be effective against malaria infections in mice.

### Example 5

### The Effect of ICI 181,551 on Growth of P. berghei in vivo

In vivo mouse experiments were done with P. berghei using a standard 4 day Peters assay. P. berghei and ICI 181,551 were administered intraperitoneally; five mice were used per data point. In IC₅₀ studies, 5 data points were taken. Mice were given 5×10⁷ parasites on day 0, and indicated concentrations of drug twice a day on days 0-3. Tail bleeds were carried out on day 4 to ascertain parasitemia, after which the animals were sacrificed.

### Results

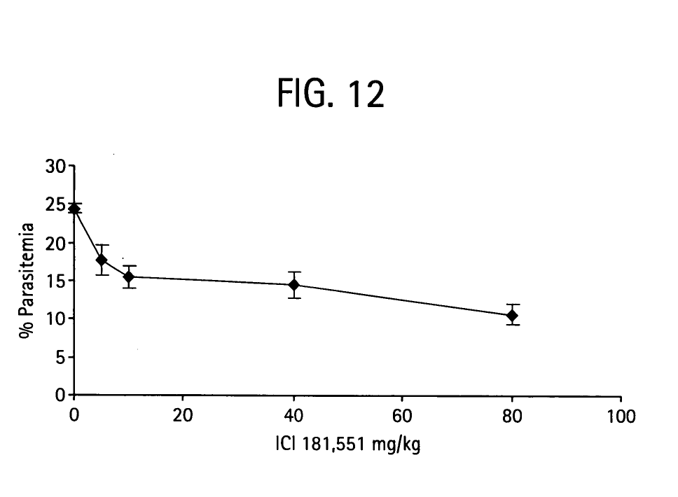

The results, as shown in FIG. 12, demonstrate that ICI 181,551 is effective at inhibiting parasitemia in vivo.

### Example 6

### Comparison of the Antagonists Racemic Propranolol, ICI, Altenolol and Nadolol in vivo Using P. berghei

In vivo mouse experiments were done with P. berghei using a standard 4 day Peters assay. P. berghei and the indicated antagonists were administered intraperitoneally; five mice were used per data point. Mice were given 5×10⁷ parasites on day 0, and indicated concentrations of drug twice a day on days 0-3. Tail bleeds were carried out on day 4 to ascertain parasitemia, after which the animals were sacrificed.

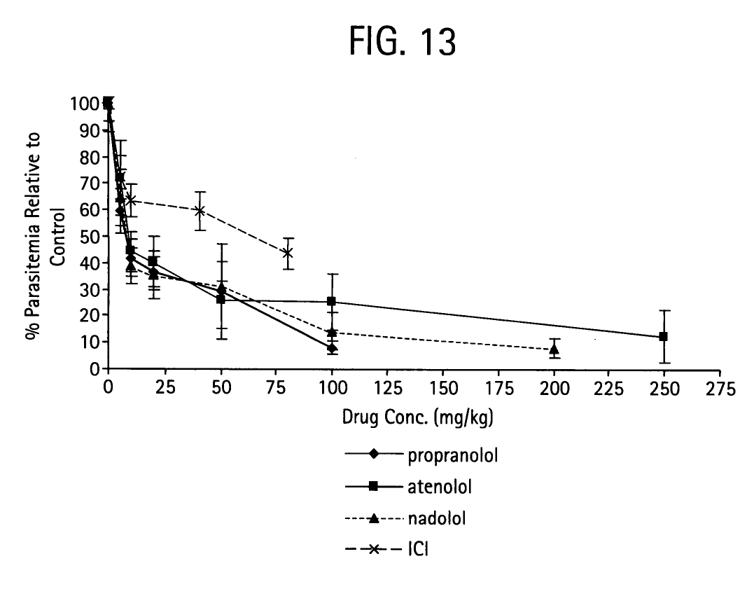

The results, as shown in FIG. 13, demonstrate that various antagonists are effective at inhibiting parasitemia in vivo.

1. A method for treating a mammal suffering from malaria or the anemia associated with a malarial infection or the sequelae of malarial infection, comprising administering a therapeutically effective amount of a compound that down-regulates G protein signaling in cells susceptible to infection by malaria.

2. The method of claim 1, wherein the compound comprises an antagonist or an inverse agonist of a G protein receptor.

3. The method of claim 2, wherein the receptor is a β-adrenergic receptor.

4. The method of claim 2, wherein the antagonist or inverse agonist is selected from the group consisting of Acebutolol, Atenolol, Betxolol, Bisoprolol, Esmolol, Metoprolol, Carteolol, Nadolol, Penbutolol, Pindolol, Propranolol, Sotalol, Timolol, Carvedilol, Labetalol, Alprenolol, and ICI 118,551.

5. The method of claim 2, wherein the receptor is an adenosine receptor.

6. The method of claim 2, wherein the antagonist is 8-SPT.

7. A method for preventing malarial infection in a mammal comprising administering a therapeutically effective amount of a compound that down-regulates G protein signaling in cells susceptible to infection by malaria.

8. The method of claim 7, wherein the compound comprises an antagonist or an inverse agonist of a G protein receptor.

9. The method of claim 8, wherein the receptor is a β-adrenergic receptor.

10. The method of claim 8, wherein the antagonist or inverse agonist is selected from the group consisting of Acebutolol, Atenolol, Betxolol, Bisoprolol, Esmolol, Metoprolol, Carteolol, Nadolol, Penbutolol, Pindolol, Propranolol, Sotalol, Timolol, Carvedilol, Labetalol, Alprenolol, and ICI 118,551.

11. The method of claim 8, wherein the receptor is an adenosine receptor.

12. The method of claim 8, wherein the antagonist is 8-SPT.

13. A method for ameliorating the symptoms associated with a malarial infection in a mammal, comprising administering a therapeutically effective amount of a compound that down-regulates G protein signaling in cells susceptible to infection by malaria.

14. The method of claim 13, wherein the compound comprises an antagonist or an inverse agonist of a G protein receptor.

15. The method of claim 14, wherein the receptor is a β-adrenergic receptor.

16. The method of claim 14, wherein the antagonist or inverse agonist is selected from the group consisting of Acebutolol, Atenolol, Betxolol, Bisoprolol, Esmolol, Metoprolol, Carteolol, Nadolol, Penbutolol, Pindolol, Propranolol, Sotalol, Timolol, Carvedilol, Labetalol, Alprenolol, and ICI 118,551.

17. The method of claim 14, wherein the receptor is an adenosine receptor.

18. The method of claim 14, wherein the antagonist is 8-SPT.

19. The method of any of claims 1, 7 or 13, wherein the administering comprises a combination of compounds that down-regulate G protein signaling in cells susceptible to infection by malaria.


SEQUENCE LISTING (4 sequences)

SEQ ID NO: 1 - protein, 11 aa, Artificial Sequence
  synthetic G alpha s peptide
QRMHLRQYELL

SEQ ID NO: 2 - protein, 11 aa, Artificial Sequence
  synthetic scrambled G s peptide Gscb1
ELRLQHYMQLR

SEQ ID NO: 3 - protein, 11 aa, Artificial Sequence
  synthetic C terminal peptide of Gq
LQLNLKEYNLV

SEQ ID NO: 4 - protein, 11 aa, Artificial Sequence
  synthetic reverse C terminal peptide of Gri
NGIKCLFNDKL